(19) United States
(12) Patent Application Publication
(10) Pub. No.: US 2026/0314970 A1
(43) Pub. Date: Oct. 15, 2026

(54) METHOD AND SYSTEM FOR MEASURING BRAIN REFLEXES AND THE MODULATORY EFFECT OF ENGAGEMENT AND LIFESTYLE

(71) Applicant: Blinklab, Princeton, NJ (US)

(72) Inventors: Peter BOELE, Princeton, NJ (US); Anton UVAROV, Princeton, NJ (US); Henk-Jan BOELE, Princeton, NJ (US)

(73) Assignee: Blinklab, Princeton, NJ (US)

(21) Appl. No.: 19/476,252

(22) PCT Filed: Apr. 19, 2024
(86) PCT No.: PCT/US2024/025344

§ 371 Date: Oct. 17, 2025

Related U.S. Application Data
(60) Provisional application No. 63/460,541, filed on Apr. 19, 2023.

(51) Int. Cl. A61B 5/16; A61B 5/00; A61B 5/11

(52) U.S. Cl. CPC A61B 5/165; A61B 5/1103; A61B 5/1126; A61B 5/163; A61B 5/6803; A61B 5/7271; A61B 5/7405; A61B 5/7445; A61B 2560/0462; A61B 2560/0487

(57) ABSTRACT

The present disclosed relates to a method and system for measuring emotional engagement in response to auditory and/or visual stimuli using, e.g., auditory startle responses, prepulse inhibition, and spontaneous eye blink and/or eye movements. The method may include displaying a first video or picture content. While an individual is watching the first video or picture content, capturing second video including one or more eyes of the individual, and while capturing the second video, exposing the individual to one or more visual and/or auditory stimuli. The method may include determining values representing eye closure based on the second video. The method may include calculating a metric based on the values.

### CROSS-REFERENCE TO RELATED APPLICATIONS

The present application claims priority to U.S. Patent Provisional Application No. 63/460,451, filed Apr. 19, 2023, the contents of which is incorporated by reference herein in its entirety.

### TECHNICAL FIELD

The present disclosure is drawn to techniques for determining emotional engagement and lifestyle and physical exercise, and specifically to determining the effects of these on auditory startle responses, prepulse inhibition, and spontaneous and anticipatory eye blink and eye movements.

### BACKGROUND

This section is intended to introduce the reader to various aspects of art, which may be related to various aspects of the present invention that are described and/or claimed below. This discussion is believed to be helpful in providing the reader with background information to facilitate a better understanding of the various aspects of the present invention. Accordingly, it should be understood that these statements are to be read in this light, and not as admissions of prior art.

Emotional engagement is an important factor in assessing the effectiveness of visual stimuli such as videos or pictures. Lifestyle and physical exercise have repeatedly been reported to have advantageous effects on brain functions, including learning. However, objective tools to measure such effects are often lacking. Eyeblink conditioning is a well-characterised method for studying the neural basis of associative, procedural learning. As such, this paradigm has potential as a tool to assess to what extent exercise affects one of the most basic forms of learning. Until recently, however, using this paradigm for testing human subjects in their daily life was technically challenging. As a consequence, no studies have investigated how exercise affects eyeblink conditioning in humans.

Traditional methods of measuring emotional engagement and lifestyle on brain function have relied on self-report measures or observer ratings, which can be subjective and unreliable. Physiological measures have been proposed as an objective way to assess emotional engagement. Among the physiological measures, auditory startle responses, prepulse inhibition, and spontaneous and anticipatory eye blink and eye movements are widely used. However, there is a need for a convenient and accessible system that allows remote determination of these measures.

### BRIEF SUMMARY

Various deficiencies in the prior art are addressed below by the disclosed systems and techniques.

In various aspects, a method for measuring emotional engagement while watching video or picture content may be provided. The method may include displaying, on a display, a first video or picture content. The method may include, while an individual is watching the first video or picture content on the display: 1) using a camera to capture second video including one or more eyes of the individual; and 2) while capturing the second video, exposing the individual to one or more visual and/or auditory stimuli. The method may include determining values representing eye closure based on the second video. The method may include calculating a metric (such as cognitive load, effectiveness of training, etc.) based on the values. In some embodiments, the metric may be emotional engagement (e.g., with the first video), and the metric may be determined based on an eyelid startle response. In some embodiments, the metric may be the impact of physical activity, and the metric may be determined based on eyeblink conditioning.

All steps of the method may be performed on a single local device (such as a desktop computer, laptop computer, mobile phone, or tablet). Some of the steps may be performed using one or more remote processing units. For example, the steps of determining values and calculating the metric may be performed by one or more remote processing units.

The method may include receiving first information from one or more remote processing units, the first information including the first video or picture content. The first information may also include information related to the stimuli to expose the individual to (e.g., the one or more visual and/or auditory stimuli, values representative of the one or more visual and/or auditory stimuli, or both). The method may include sending second information to one or more remote processing units, the second information including the second video.

The display and camera may be operably coupled to a headset (such as a virtual reality (VR) headset, or an augmented reality (AR) or mixed reality (MR) headset). The headset may be operably coupled to one or more processing units performing the method.

Calculating the metric may be performed by a trained machine learning algorithm that has been trained using categorized videos and/or picture content. The metric may be based at least partially on detected alpha startle responses.

The method may include displaying the metric to the individual.

In various aspects, a system for measuring emotional engagement while watching video or picture content may be provided. The system may include a display, a camera, a speaker, a memory, one or more processing units operably coupled to the display, camera, speaker, and memory, and a non-transitory computer-readable storage medium. The storage medium may include instructions that, when executed by the one or more processing units, cause the one or more processing units to, collectively, perform the disclosed method.

The system may be configured as a desktop computer, laptop computer, mobile phone, or tablet. The one or more processing units may include one or more local processing units and one or more remote processing units. The one or more remote processing units may be configured to perform one or more steps of the method. For example, the one more processing units may, collectively, determine values and calculate the metric.

The display and camera may be operably coupled to a headset (such as a virtual reality (VR) headset, or an augmented reality (AR) or mixed reality (MR) headset). The headset may be operably coupled to one or more processing units performing the method.

### BRIEF DESCRIPTION OF FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a schematic illustration of a system.

FIG. 2 is an illustration of a headset.

FIG. 3 is a flowchart of a method.

FIG. 4 is an illustration of a template for tracking facial landmarks, and eye landmarks in particular.

FIGS. 5A-5B are graphs showing exemplary normalized eyelid closure data as determined by an AI analyzing facial images of a user watching videos categorized as negative, neutral, or positive, when exposed to only a loud sound (5A), or a soft sound followed by a load sound (5B).

FIGS. 5C-5D are graphs showing eyelid closure of alpha startles as determined by an AI analyzing facial images of groups of users watching videos categorized as negative, neutral, or positive, when exposed to only a loud sound (5C), or a soft sound followed by a loud sound (5D).

FIG. 6A shows graphs of conditioned response (CR) amplitude by session for paired (CS+US) and CS-only trials combined in the sedentary and active groups with or without exercise before eyeblink conditioning sessions. Active individuals showed significant conditioning with the post-exercise group showing significantly higher conditioned response amplitudes at sessions 1 and 2 compared to the no exercise group. shading represents standard error of the mean.

FIGS. 6B and 6C shows graphs for sedentary (6B) and active (6C) group averaged eyelid traces for paired (CS+US) trials (top panels) and CS-only trials (bottom panels) without (left panels) or after (right panels) exercise for three eyeblink conditioning sessions. Lightly shaded blocks indicate the presentation of the CS for 450 ms and darker shaded blocks indicate the actual (US) or expected (US omitted) presentation of the US for 50 ms co-terminating with the CS at 450 ms. In paired trials, note the peak in amplitude following the presentation of the US, namely the unconditioned response (UR) present in all groups. Note the shift in timing of the rise in amplitude in paired trials to precede the presentation of the US at later sessions—CR—especially obvious in the active, post-exercise group. The acquisition of conditioned responses over the three sessions is also illustrated by the rise in amplitude in the CS-only trials, again particularly obvious in the active, post-exercise group. Significance levels: * p<0.05, ** p<0.01; n.s.=not significant.

FIGS. 7A and 7B are graphs showing distribution of latency to conditioned response peak for all conditioned stimulus (CS) only trials across all sessions in sedentary (7A) and active (7B) groups with or without exercise. The darker shaded block at 400 ms indicates the expected onset of the unconditioned stimulus (omitted US) which is omitted in these trials. The lighter shaded block indicates the presentation of the CS. Note the distribution centred roughly around the expected onset of the US at 400 ms for all groups.

FIGS. 7C and 7D are boxplots of percentage of well-timed conditioned responses (CRs) in the sedentary (7C) and active (7D) groups with or without exercise. Middle line indicates group medians, box ends indicate lower and upper quartiles, whiskers indicate group minima and maxima and dots indicate outliers. n.s.=not significant.

FIGS. 8A and 8B are graphs and box-plots showing group averaged unconditioned response amplitudes for sedentary (8A) or active (8B) individuals with (solid line) or without (dashed line) exercise preceding the eyeblink conditioning session. Unconditioned response amplitudes were calculated for the first two blocks of session 1, prior to the development of conditioned responses. The darker shaded block (top panels) from 400-450 ms indicates the presentation of the unconditioned stimulus (US). Note that the unconditioned response amplitude was similar regardless of the exercise condition for both sedentary and active groups. In the boxplots (bottom panels), the middle line indicates group medians, box ends indicate lower and upper quartiles, whiskers indicate group minima and maxima. n.s.=not significant.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

The present disclosure provides a method and system for measuring the impact or effectiveness of an activity based on a measure of brain reflexes. For example, one can measure emotional engagement in response to visual stimuli or effectiveness of a physical activity based on the brain reflexes. Said differently, the disclosed techniques can be used to correlate the determined values relating to eye movement and eye blinks to various metrics of interest.

Brain reflexes are basic and unconscious responses that can be used as indicators of the functional integrity of the nervous system. An important reflex is the acoustically evoked eyelid startle reflex, which has been studied for more than fifty years. Moreover, the startle reflex can serve as an effective unconditioned stimulus (US) in Pavlovian eyeblink conditioning, which is a well-known method for studying the neural correlates of procedural learning and memory. In eyeblink conditioning, a US that reliably evokes a reflexive eyeblink, is repeatedly paired with a conditioned stimulus (CS). Eventually the CS itself will evoke an anticipatory eyeblink, which is called a conditioned response (CR).

Any appropriate CS and/or US may be utilized. This may include one or more visual stimuli, such as a particular video, image, or flash of light (such as a front-facing camera flash, or even an all-white image being displayed on a screen), etc. This may include one or more auditory stimuli, such as a tone generated at one or more frequencies, white noise, etc.

Previously, eyeblink conditioning was relatively difficult to assess in human participants. As a result, studies using neurobehavioral assays to measure the effects of lifestyle interventions on learning in human subjects are limited.

In some embodiments, the auditory and/or visual stimuli may be configured to generate a startle response. In some embodiments, rather than attempting to generate a conditioned response, only an unconditioned stimulus may be utilized.

Similarly, prepulse inhibition (PPI) may be utilized. PPI is the behavioral phenomenon whereby the magnitude of the startle response is inhibited when a short and loud startling stimulus (the pulse, such as a loud sound) is preceded by a weaker stimulus that does not elicit a startle reflex (the prepulse, such as a quieter sound). Herewith, PPI measures sensorimotor gating, which is the mechanism of the nervous system to filter out irrelevant sensory information to protect the brain from overstimulation and enabling appropriate reaction to stimuli that are relevant. PPI is less brain region specific and probes midbrain function and modulatory effects that the midbrain receives from limbic systems, thalamus, and prefrontal areas.

In various aspects, a system for measuring impact or effectiveness of an activity may be provided.

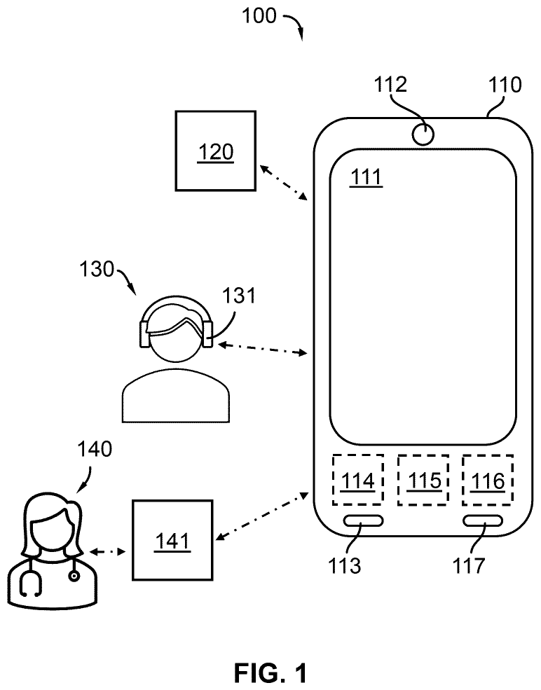

Referring to FIG. 1, the system (100) may include one or more devices (110). The system may include a display (111), a camera (112), a speaker (113), a memory (114), one or more processing unit(s) (115), and a non-transitory computer-readable storage medium (116). In some embodiments, the system may include a microphone (117). The storage medium may include instructions that, when executed by the one or more processing units, cause the one or more processing units to, collectively, perform specific steps of a method.

As used herein, the term “processing unit” generally refers to a computational device capable of accepting data and performing mathematical and logical operations as instructed by program instructions. This may include any central processing unit (CPJ), graphics processing unit (GPU), core, hardware thread, or other processing construct known or later developed. The term “thread” is used herein to refer to any software or processing unit or arrangement thereof that is configured to support the concurrent execution of multiple operations.

The system may be configured as (or may include) a desktop computer, laptop computer, mobile phone, or tablet.

In some embodiments, only the processing units on the device (e.g., on a smartphone, etc.) are utilized. In some embodiments, one or more steps may be performed by remote processing units. For example, the one or more processing units may include one or more local processing units (e.g., processing unit(s) (115)) and one or more remote processing units (e.g., remote processing unit(s) (120) and/or remote processing unit(s) (141)). Aside from steps that necessarily require interaction with a user (130), the various steps may be distributed in any manner between local and remote processing units. For example, in some embodiments, the one more processing units may, collectively, determine values and calculate the metric. In some embodiments, remote processing unit(s) (120) may be a cloud-based processing unit. In some embodiments, remote processing unit(s) (141) may be configured to receive and/or display information to a remote user (140), e.g., a clinician, doctor, researcher, etc.

In some embodiments, the system may include headphones (131) for a user (130) to wear.

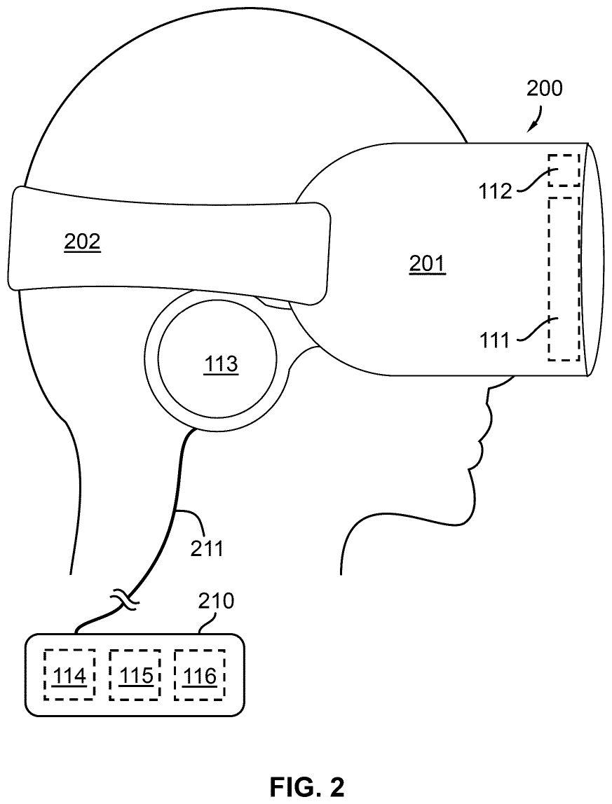

Referring to FIG. 2, in some embodiments, the display (111) and camera (112) may be operably coupled to a headset (200). The display and camera may be disposed within a headset housing (201). The headset may be a virtual reality (VR) headset (e.g., a headset that provides a fully virtual experience, where the user can only see the display provided in the headset), an augmented reality (AR) headset (e.g., a headset providing a live or near-live image of the physical world captured by a camera into which a computer-generated object or objects are superimposed so as to appear to be a part of the physical world when the live or near-live image and the object or objects are displayed on a screen. A display screen or other controls may cause the augmented reality to adjust as changes to the captured images of the physical world indicate updated perspectives of the physical world), or a mixed reality (MR) headset (a headset means a combined virtual objects and spaces and physical reality objects. It is closely related to augmented reality but may include, for example, a projection of an actual image of a person who is in a different physical location, using cameras to capture that person's image, then superimposing that person within a different physical environment using augmented reality).

As shown in FIG. 2, the headset may have a strap (202) configured to hold the headset on a user's head. The headset may be operably coupled to one or more processing units performing the method, either wirelessly or wired. In FIG. 2, a wire (211) is used to couple the headset (200) to a housing (210) containing the memory (114), processing unit(s) (115), and non-transitory computer-readable storage medium (116).

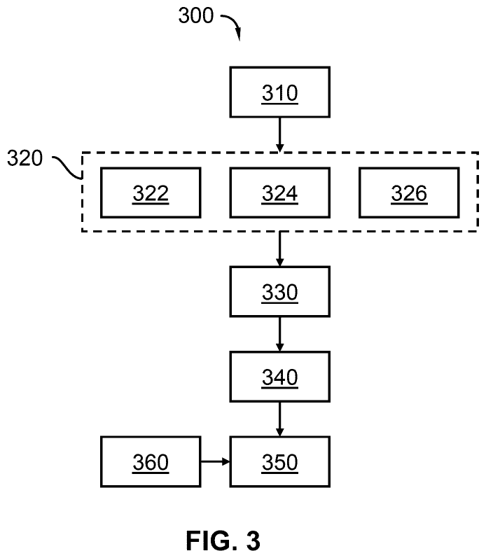

The processing unit(s) may be configured to collectively perform various steps of a method. Referring to FIG. 3, the method (300) may optionally include receiving (310) first information from one or more remote processing units. The first information may include information defining or relating to a video or image that may be displayed to a user. In some embodiments, the video or image to be displayed is what is received. For example, a researcher could send a video or image directly to a user's device, or the research could send a URL to a user's device, after which the device could process that URL and download a video or image found at the URL, storing it for later use. The researcher could also send information stating the length and intensity of any prepulses or pulses used for stimuli. In some embodiments, the video or image to be displayed is randomly determined.

The first information may also include information related to the stimuli to expose the individual to (e.g., the one or more visual and/or auditory stimuli, values representative of the one or more visual and/or auditory stimuli, or both).

The method may include testing (320) brain reflexes of a user. This may be done by displaying (322), on a display (e.g., display (111)), a first video or picture content (which may be the video or image received in the receiving (310) step, or may be an image or video already on the device.

The testing may include, while an individual is watching the first video or picture content on the display, capturing (324) (e.g., with camera (112)) a second video including one or more eyes of the individual.

The testing may include, while capturing the second video, exposing (326) the individual to one or more visual and/or auditory stimuli. Any appropriate visual or auditory stimuli may be utilized. In some embodiments, a camera flash or causing the display to flash bright white for a brief amount of time may be used as a visual stimulus. In some embodiments, a tone, such as a beep, or white noise, may be used as an auditory stimulus. In some embodiments, only visual or auditory stimuli are used. In some embodiments, both visual and auditory stimuli are used.

The second video may capture video for a period of time before the stimuli, during the stimuli, and for a period of time after the stimuli. The period of time after the stimuli may be up to 500 ms after the stimuli.

The method may optionally include sending (330) second information to a remote processing unit, the second information including the second video.

The method may include determining (340) values representing eye-related movements, such as eye closure, based on the second video. This may also include determining values representing blink amplitude, blink duration, and blink timing based on the second video including one or more eyes of the individual. Blinks elicited by the presentation of a blink-evoking stimulus, such as an unexpected loud sound or visual stimulus, may be determined. In addition, spontaneous blinks may be determined.

In some embodiments, the eye-related movements may include a spontaneous eye blink. In some embodiments, the eye-related movements may include a reflex eye blink. In general, spontaneous blinks occur without any external stimuli and/or internal effort, while reflex blinks typically occur in response to external stimuli. One type of reflex blink is an anticipatory eye blink, that may be developed during eyeblink conditioning.

In some embodiments, the eye-related movements may include eye position tracking. The eye position tracking may include the tracking of (i) fast eye movement (saccades and micro-saccades), (ii) smooth pursuit movements, and/or (iii) vestibulo-ocular movements. In preferred embodiments, if eye position tracking is utilized, the device is configured to utilize a VR-type viewer as described herein.

In some embodiments, the eye-related movements may include pupil size tracking to measure the user's alertness. As is known in the art, pupil size decreases as alertness wanes. By analyzing captured images in order to measure the pupil diameter, and optionally normalizing them, the pupil size can be tracked over time in order to determine if the user is sufficiently alert. In some embodiments, a level of alertness is determined by comparison the pupil size to other pupil size measurements gathered during the user's testing. In some embodiments, a level of alertness is determined by comparing a measured pupil size to a threshold.

In some embodiments, the eye pupil size tracking may be used to measure conditioned pupil responses. This is similar to eyeblink conditioning, but where the pupil size is measured instead of the eyelid position. That is, an image is captured containing the pupil, the pupil diameter is measured, and preferably normalized, after experiencing conditional and unconditional stimuli, just as is done using FEC for eyeblink conditioning.

For example, computer vision and image processing techniques may be used to detect fully automated and real-time landmarks on a human face. More preferably, the algorithm is optimized to provide fast and accurate tracking of eyelids in both adults and infants. Any appropriate technique known to train a machine-learning algorithm can be utilized here.

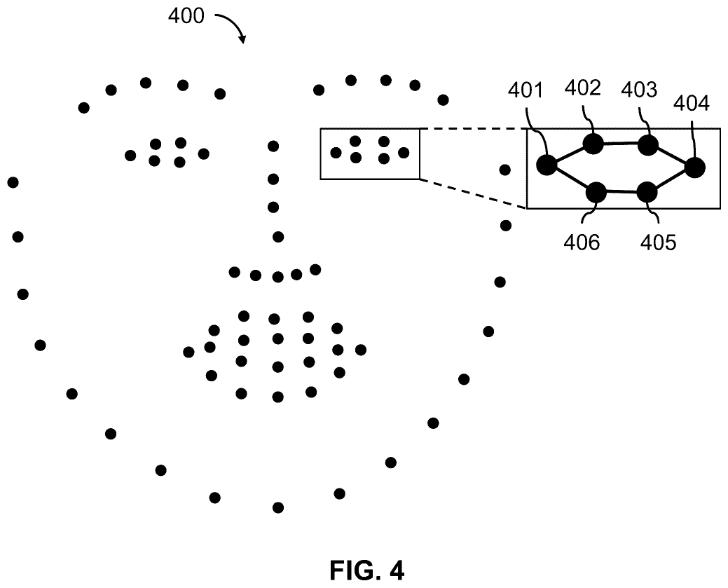

An algorithm may be used to detect a plurality of landmarks on the face. In FIG. 4, an example of a template (400), using 68 landmarks, is shown. In some embodiments, the template (400) may comprise or consist of 6 landmarks for each eye captured in the image. The six landmarks are, as seen in FIG. 4, a left corner (401), an upper left eyelid mark (402), an upper right eyelid mark (403), a right corner (404), a bottom right eyelid mark (405), and a bottom left eyelid mark (406). As will be readily understood by those of skill in the art, other templates and/or mesh models can easily be incorporated here; this is merely a simplified example.

Once the landmarks are identified, calculations can be made. As an example of this, for each image, a Fraction Eyelid Closure (FEC) can be calculated. Using the preferred six landmarks as an example, conceptually, the calculation is made by looking at the differences in position of the six marks, and in particular:

$$FEC = \frac{\begin{matrix}( ‖ Upper Left ( 402 ) - Lower Left ( 406 ) ‖ + \\ ‖ Upper Right ( 403 ) - Lower Right ( 405 ) ‖ )\end{matrix}}{2 \times ( ‖ Left Corner ( 401 ) - Right Corner ( 404 ) ‖ )} \qquad ( 1 )$$

When looking at multiple images of the same individual, a normalized (“FEC_{NORM}”) can be determined, based on the minimum FEC (“FEC_{MIN}”) and maximum FEC (“FEC_{MAX}”). Specifically, FEC_{NORM}=1−(FEC−FEC_{MIN})/(FEC_{MAX}). An FEC_{NORM} of 0 corresponds to an eye that is fully open, and an FEC_{NORM} of 1 corresponds to an eye that is fully open.

As will be understood in the art, while use of FEC is described, other known techniques for determining values representing eye-related movements may be utilized as appropriate. For example, the Apple ARKit's blend shape coefficients and MediaPipe can provide coefficients (generally values from 0.0 to 1.0) for detected facial expressions, including right and left eye blink closures (eyeBlinkRight and eyeBlinkLeft, respectively).

In some embodiments, where two eyes are detected, various techniques may be used. An FEC may be calculated for each eye and the results may be, e.g., averaged together (or otherwise statistically combined). An FEC may be calculated for each eye, and the minimum value may be utilized. An FEC may be calculated for each eye, and the maximum value may be utilized. An FEC may be calculated for each eye, and a difference between the two FEC values may be determined. If the difference is above a threshold, the value of a flag may be set to 1 or a variable may be increased, indicating an anomalous response occurred.

In some embodiments, if no eyes are detected in a given image, or more than two eyes are detected, the image may be skipped.

A calibration sequence may have occurred prior to these steps, and FEC_{MIN} and FEC_{MAX} values may be determined based the images or video captured during calibration. In some embodiments, FEC_{MIN} and FEC_{MAX} values may be determined based solely on the images or video captured as part of the testing described above.

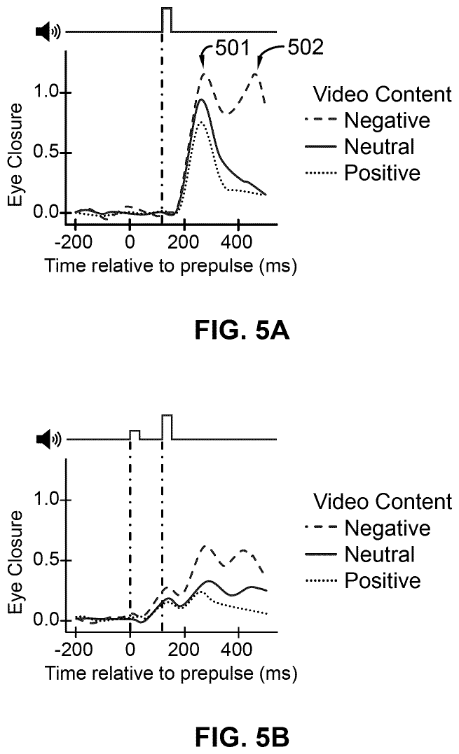

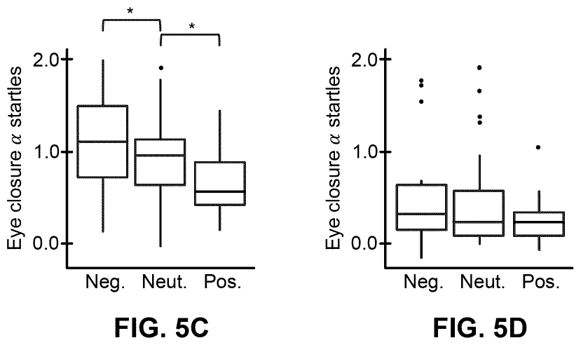

Referring briefly to FIG. 5A, when a user has been exposed to a stimulus (such as an unexpected loud sound) the individual may close their eyes to some extent. There may be an alpha startle (501) in response to the loud noise. There may also be a beta startle (502) response that appears some time after the alpha startle.

The method may include calculating (350) a metric based on the values.

Calculating the metric (such as cognitive load) may be performed by a trained machine learning algorithm that has been trained using categorized videos and/or picture content. Thus, in some embodiments, the method may include training (360) the machine learning algorithm.

In some embodiments, the metric may be calculated by comparing the value representing the eye-related movement to a calibration curve or to predetermined threshold ranges. These calibration curves or threshold ranges may be specific to the individual, or may be a generic calibration curve or threshold range that applies to multiple users.

As an example, for emotion engagement, in some embodiments the calibration curve or threshold ranges may be determined by showing a user (or a plurality of users) a plurality of randomized videos or images. The plurality of randomized videos or images may include at least one video or image that is known to have a positive valence (e.g., a calming video or a cute image) and at least one video or image that is known to have a negative valence (e.g., an upsetting image or a video that generates fear). During each video or image during calibration, the users are exposed to auditory and/or visual stimuli (preferably the same stimuli intended to be used during normal testing), and the eye-related movements to the stimuli are detected and measured. After sufficient values representing eye-related movements have been determined for the plurality of videos in the calibration sequence, a calibration curve or threshold range can be determined. That calibration curve or threshold range can then be used to correlate the eye-related movement to the degree that a test video or image generates an emotional (positive or negative) response in the user.

In some embodiments, the metric may be based at least partially on detected alpha startle responses. In some embodiments, the metric may be based at least partially on detected beta startle responses.

In some embodiments, the metric may be emotional engagement (e.g., with the first video), and the metric may be determined based on an eyelid startle response. In some embodiments, the metric may be the impact of physical activity, and the metric may be determined based on eyeblink conditioning.

FIGS. 5A-5D are graphs related to an example where participants were shown short video clips on, e.g., a smartphone with either neural, positive, or negative valence, and then exposed to a pulse and optionally a pre-pulse of stimuli, the stimuli being a bright light (camera flash) and a loud noise (white noise). Video clips were taken from the Database of Emotion Videos from Ottawa (DEVO). Statistically significant differences between the three valences can be detected in terms of the degree of eye closure experienced when exposed to a loud noise with (FIGS. 5B, 5D) or without (FIGS. 5A, 5C) a pre-pulse. Further, it was noted that watching a happy video, if the user is emotionally engaged, they will become less responsive to the stimuli than if they are not emotionally engaged. If the user is less emotionally engaged, they are more responsive. Additionally, if the video is scary, an emotionally engaged user will evoke an anxiety response.

Thus, the eye closure amount (and/or blink rate, auditory startle responses, etc.) may be used to determine a metric (such as emotional engagement, sufficiency of exercise, etc.). In some embodiments, those determine metric(s) may be used to determine an additional metric. For example, a score for a video may be determined that is an average of the emotional engagement determined by the eye closure amount across a plurality of individuals who watched the video. Thus, by exposing a group of individuals to a plurality of videos or images, and exposing those individual to a stimuli while they are watching the videos or images, it is possible to determine which video or image generated the most emotional engagement.

All steps of the method may be performed on a single local device (such as a desktop computer, laptop computer, mobile phone, or tablet). Some of the steps may be performed using one or more remote processing units. For example, the steps of determining values and calculating the metric may be performed by one or more remote processing units.

In some embodiments, the instructions on the storage medium may cause the processing unit(s) to include two parts or modules: a testing module and an analysis module.

The testing module presents visual stimuli to the participant and records the physiological responses (see FIG. 3, testing (320) step). As disclosed herein, the visual stimuli may be, e.g., videos, pictures, or any other type of visual content. The physiological responses that may be recorded include, e.g., auditory startle responses, prepulse inhibition, and spontaneous eye blink and eye movements. All these responses may be measured by the analysis module during execution of the method.

The present disclosure provides a convenient and accessible system for measuring emotional engagement in response to visual stimuli. The system allows remote testing and analysis, which makes it suitable for use in a variety of settings, including research, marketing, and clinical applications.

The use of auditory startle responses, prepulse inhibition, and spontaneous eye blink and eye movements provides an objective and reliable measure of various metrics that may be useful, e.g., for the benefit of the individual being tested such as for self-improvement or diagnostic purposes. The effects of a physical activity can be measured, for example, to determine if the physical activity was effective, if the level of activity was sufficient to provide a detectable benefit, etc. Alternatively, this may include being able to quantify emotional engagement, which can be used to optimize the effectiveness of visual stimuli.

### Example 1

### Participants

40 Neurotypical participants aged between 18 and 40 years, were recruited by social media invitations to participate in the study. This sample size is in-line with other eyeblink conditioning research in humans. Participants were divided into an active or sedentary group based on their weekly hours of physical activity. The cut-off point for group classification was determined using the lower limit of the WHO guidelines for physical activity in adults aged 18-64 years. Participants doing less than 2.5 hours of moderate intensity or less than 75 minutes of vigorous intensity exercise were in the sedentary group and the other participants were in the active group. Moderate intensity was defined as: “Exercise that increases heart rate but you are still able to hold a conversation” and vigorous as “Exercise that raises your heart rate so that you are unable to speak”. Education level was similar across groups as all subjects either had a university degree or were university students. Furthermore, the average age and hours of sleep per night were similar across groups (see Table 1).

TABLE 1
Demographic and exercise-related data for participants in the active, post-exercise;
active, no-exercise; sedentary, post-exercise; and sedentary, no-exercise groups.

| | Active, | Active, | Sedentary, | Sedentary, | | |
|---|---|---|---|---|---|---|
| | post-exercise | no-exercise | post-exercise | no-exercise | | |
| | (n = 9) | (n = 9) | (n = 9) | (n = 9) | Statistical | |
| | Mean (±SD) | | | | test | p-value |
| Moderate | 3.2 | 4.7 | 0.9 | 1.1 | F_{3, 32} = 12.5 | <0.0001 |
| exercise | (±1.6) | (±2.4) | (±0.8) | (±0.8) | | |
| (hours/week) | | | | | | |
| Vigorous | 4.1 | 2.1 | 0.2 | 0.0 | F_{3, 29} = 9.6 | 0.0001 |
| exercise | (±3.1) | (±1.3) | (±0.3) | (±0.0) | | |
| (hours/week) | | | | | | |
| Total | 7.3 | 6.8 | 1.1 | 1.1 | F_{3, 32} = 20.3 | <0.0001 |
| exercise | (±3.4) | (±2.9) | (±0.8) | (±0.8) | | |
| (hours/week) | | | | | | |
| Age (years) | 24.9 | 25.6 | 22.2 | 24.9 | F_{3, 32} = 1.01 | 0.40 |
| | (±2.3) | (±5.9) | (±3.6) | (±5.0) | | |
| Sleep | 7.5 | 7.9 | 7.6 | 7.3 | F_{3, 30} = 0.8 | 0.49 |
| (hours/night) | (±0.8) | (±0.3) | (±1.2) | (±0.4) | | |
| Self-rated | 2.8 | — | 3.7 | — | t_{11, 51} = −2.30 | 0.04 |
| exercise | (±0.5) | | (±1.0) | | | |
| intensity/5 | | | | | | |

Sex

| | | | | | | |
|---|---|---|---|---|---|---|
| Male | 4 | 4 | 3 | 5 | — | — |
| | (44.4%) | (44.4%) | (33.3%) | (55.6%) | | |
| Female | 5 | 5 | 6 | 4 | — | — |
| | (55.6%) | (55.6%) | (66.7%) | (44.4%) | | |

### Experiments

Experiments were conducted via a smartphone application. During the experiment, participants watched audio-normalised nature documentaries (n=109 views) or TV shows: The Office (n=6 views), Modern Family (n=3 views), or Coco (n=2 views). A delay eyeblink conditioning paradigm, a form of cerebellar associative learning, was used in this study. The eyeblink conditioning experiment consisted of the pairing of a CS with a US (here, a burst of white noise plus activation of the camera's selfie flash). The CS (here, a white dot) was presented in the centre of the phone screen for 450 ms. In paired trials, the US was presented 400 ms after the onset of the CS and co-terminated with the CS. In US-only trials, the stimuli were presented for 50 ms, 400 ms from trial onset. Each eyeblink conditioning session consisted of 10 blocks and a pre-block at the start of each session. The pre-block consisted of 3 CS-only trials and 2 US-only trials. Within each block, for blocks 1-10, there were 8 paired trials, 1 CS-only trial and 1 US-only trial semi-randomly distributed throughout the block.

### Experimental Setup:

Participants were instructed to use headphones and complete the experiments in a quiet, well-lit room. All participants completed four sessions of experiments in the space of a week, with no sessions done on the same day. Session 1 was an introductory session where participants were supervised remotely and became familiar with the experimental setup. This session did not include the eyeblink conditioning paradigm. For conciseness, this example will only focus on the eyeblink conditioning paradigm and from here sessions 2-4 will be referred to as sessions 1-3; session 1 being the first eyeblink conditioning session.

### Exercise Groups:

Participants in the active and sedentary groups were randomly assigned to an exercise and no-exercise group. Participants in the exercise condition were instructed to do all eyeblink conditioning sessions as soon as possible after at least 30 minutes of moderate intensity running or cycling. Participants in the no-exercise group were instructed to refrain from exercise for at least 8 hours before the test. Before starting the eyeblink training session, participants were asked, in the app, to rate the intensity of the exercise on a five-point Likert type scale (see Table 1).

### Data Processing

Data processing was done in R 4.3.1. Trials were baseline corrected using the 500 ms stimulus-free baseline and min-max normalised using spontaneous blinks as a reference. Individual eyelid traces were normalised by dividing each trace by the maximum signal amplitude of the relevant session. Thus, eyes closed corresponded to a value of 1 and eyes open to a value of 0.

Trials with extreme outliers and trials where spontaneous blinks occurred within a time window of 150 ms before, until 35 ms after stimulus presentation were excluded from further analysis. Trials were then re-baseline corrected using the same time window that was used for removal of spontaneous blinks.

A mean baseline CR amplitude per subject was determined at session 0. Session 0 was defined as the pre-block CS-only trials from session 1. CR amplitude was determined as the maximum signal amplitude value at 430 ms, for paired and CS-only trials. This time value was chosen to allow for a latency of 30 ms following the expected presentation of the US at 400 ms. There is a latency in response to the US (supplemental FIG. 2) likely due to retinal processing of the flash20.

To compare latency to CR peak between groups, CS-only trials were analysed. Here, CRs were defined as trials with a maximum signal amplitude above 0.10 in a time window ranging from 60-750 ms. Additionally, the mean percentage of well-timed CRs was calculated per group. A well-timed CR was defined as a trial with a maximum signal amplitude above 0.10 in a time window between 400-500 ms.

### Statistical Analysis

All statistical analyses and visualisations were done in R 4.3.1. Potential differences between groups in age, average weekly exercise and sleep hours were tested using a one-way ANOVA. A t-test for unequal variances was used to compare the self-reported exercise intensity levels between the active and sedentary groups who completed eyeblink conditioning after exercise.

For all other analyses, multilevel linear mixed effects (LME) models were used. These models are robust to deviations from normality and are more appropriate for the nested data structure of this study^{21,22}. In all models, ‘subject’ was used as a random effect. For CR amplitude models, a random slope for the effect of sessions across subjects was used. Data for the CR amplitude models was normalised using ordered quantile normalisation, suggested by the bestNormalize package in R, to allow for optimal model fit. Fixed effects included: ‘session’ (CR amplitude models), ‘exercise’ (between group comparisons) and ‘exercise*session’ (between group comparisons). Restricted maximum likelihood method was used to estimate model parameters. Log likelihood ratio and AIC and BIC indices were used to assess the model fit. An alpha value of p<0.05 (two-tailed) was used to determine significance. For multiple comparisons, Hochberg p-value adjustments were made to account for the number of comparisons.

### Results

### Physical Activity

Participants in the sedentary, post-exercise group completed all three sessions on average 11 minutes following at least 20 minutes of running or cycling. Two participants did not follow the exercise protocol for one session; one completed session 1 after a 40 minute gym and rowing machine session and the other completed session 2 after an hour of golf. Participants in the active, post-exercise group completed all three sessions on average 14 minutes following at least 30 minutes of running or cycling. Two participants in this group did not follow the protocol- one participant completed session 3 after a 40-minute gym cardio session; one participant completed session 2 after 25 minutes of swimming and session 3 after a 30 minute gym strength session. Both active and sedentary individuals in the no-exercise group completed all three sessions without any aerobic exercise for at least 8 hours before the test.

### Conditioning—Acquisition

In some participants the acquisition of CRs already started to occur in session 1 with the amplitude and timing of these responses improving over the course of three sessions. In contrast, some participants did not acquire CRs.

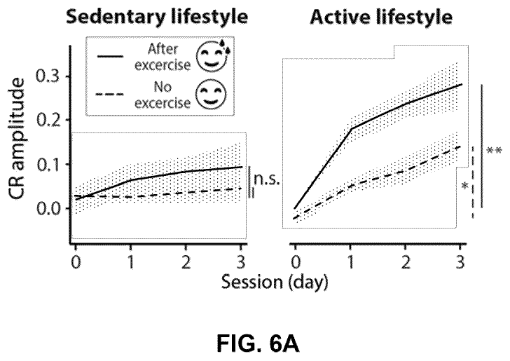

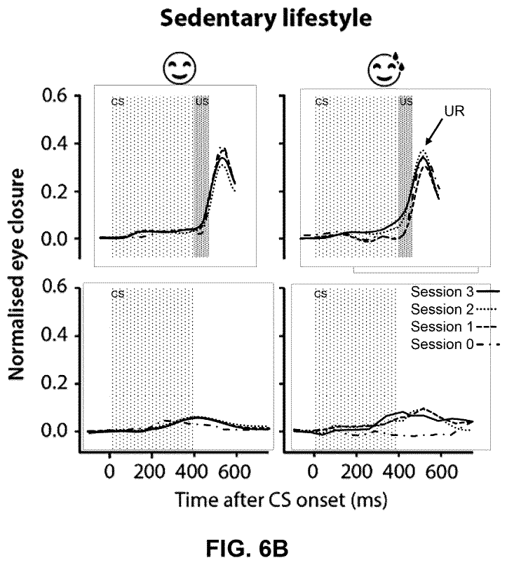

First it was determined whether there was an effect of exercise on eyeblink conditioning. CR amplitude at 430 ms was determined for each group (see FIGS. 6A-6C) and compared between groups who did the sessions without exercise and groups who did the sessions directly after exercise. The effect of ‘exercise’ (F_{1,34}=6.27, p=0.017) and ‘session’ (F_{3,7180}=5.13, p=0.0015) were significant, but not session*exercise (F_{3,7180}=2.07, p=0.10). Post-hoc tests revealed a significant difference between the no and post-exercise groups at session 2 (t₃₄=−2.87, p=0.028).

Next it was investigated whether this effect of acute exercise on eyeblink conditioning differed for active and sedentary individuals. When comparing the active and sedentary groups who completed eyeblink conditioning sessions after exercise, the effects of ‘lifestyle’ (F_{1,16}=7.61, p=0.014) and ‘session’ (F_{3,3643}=4.40, p=0.0043) were statistically significant, but not lifestyle*session (F_{3,3643}=0.56, p=0.64). Post-hoc tests revealed a significant difference in CR amplitude already at session 1 (t₁₆=2.81, p=0.037) and this difference was maintained at session 3 (t₁₆=2.62, p=0.037).

CR amplitude was then compared between no and post-exercise groups within the sedentary and active groups separately. Within the sedentary group, CR amplitudes did not differ significantly between conditions at any of the three sessions (see FIGS. 6A, 6B). In contrast, the CR amplitude differed between the no and post-exercise groups in the active group (see FIGS. 6A, 6C). The effect of ‘exercise’ (F_{1,16}=9.40, p=0.0074) and ‘session’ (F_{3,3296}=5.49, p=0.00092) were significant. The interaction between ‘exercise’ and ‘session’ was not significant (F_{3,3296}=0.95, p=0.42). Post-hoc tests showed a significant difference between the active post-exercise and no-exercise groups at sessions 1 (t₁₆=−2.70, p=0.048) and 2 (t₁₆=−3.23, p=0.021).

When the effect of session on CR amplitude was investigated for each group separately, significant effects were only seen in the active groups (see Table 2). In the sedentary, no-exercise group, the CR amplitude at session 3 (mean=0.05, ±0.18) was close to the baseline amplitude (mean=0.03, +0.09). In the sedentary, post-exercise group, the CR amplitude increased slightly from a mean of 0.02 (+0.15) at session 0 to 0.10 (+0.23) at session 3. The effect of ‘session’ was not significant (F_{3, 1907=1.71}, p=0.16).

In contrast, in the active, no-exercise group, the CR amplitude increased from −0.05 (+0.06) at baseline to 0.05 (+0.15) at session 1 and 0.14 (+0.22) at session 3. The effect of ‘session’ was significant (F_{3,1560}=3.99, p=0.0087) and post-hoc tests showed a significant difference between session 0 and all other sessions. Finally, the active, post-exercise group showed an increase in CR amplitude from 0.01 (+0.03) at baseline to 0.18 (+0.27) at session 1 and further to 0.28 (+0.32) at session 3. The effect of ‘session’ was significant (F_{3,1736}=2.86, p=0.036). Post-hoc tests showed a significant difference between session 0 and all other sessions.

TABLE 2
Conditioned response amplitudes and latencies in active
and sedentary groups before and after exercise

| | sedentary | | sedentary | | active | | active | |
|---|---|---|---|---|---|---|---|---|
| | no-exercise | | post-exercise | | no-exercise | | post-exercise | |
| | CR amplitude | | | | | | | |
| Session | Mean | SD | Mean | SD | Mean | SD | Mean | SD |
| 0 | 0.03 | 0.09 | 0.02 | 0.15 | −0.05 | 0.06 | 0.01 | 0.03 |
| 1 | 0.03 | 0.15 | 0.07 | 0.20 | 0.05 | 0.15 | 0.18 | 0.27 |
| 2 | 0.04 | 0.16 | 0.09 | 0.21 | 0.09 | 0.19 | 0.23 | 0.29 |
| 3 | 0.05 | 0.18 | 0.10 | 0.23 | 0.14 | 0.22 | 0.28 | 0.32 |
| Main effect | F_{3, 1971} = 0.96, | | F_{3, 1907} = 1.71, | | F_{3, 1560} = 3.99, | | F_{3, 1736} = 2.86, | |
| session | p = 0.41 | | p = 0.16 | | p = 0.0087 | | p = 0.036 | |
| Latency to | 455.83 | 169.76 | 451.14 | 150.79 | 466.63 | 176.32 | 495.63 | 131.48 |
| CR peak (ms) | | | | | | | | |
| Main effect | F_{1, 16} = 0.001, | | | | F_{1, 16} = 1.46, | | | |
| exercise | p = 0.97 | | | | p = 0.25 | | | |
| Percentage well- | 11.27 | 12.13 | 27.22 | 19.80 | 31.09 | 24.84 | 27.01 | 12.93 |
| timed CRs | | | | | | | | |
| Main effect | F_{1, 16} = 4.25, | | | | F_{1, 16} = 0.19, | | | |
| exercise | p = 0.056 | | | | p = 0.67 | | | |
| UR amplitude | 0.42 | 0.32 | 0.51 | 0.27 | 0.54 | 0.18 | 0.56 | 0.29 |

| | | |
|---|---|---|
| Main effect | F_{1, 14} = 0.15, | F_{1, 13} = 0.11, |
| exercise | p = 0.71 | p = 0.75 |

### Conditioning Timing

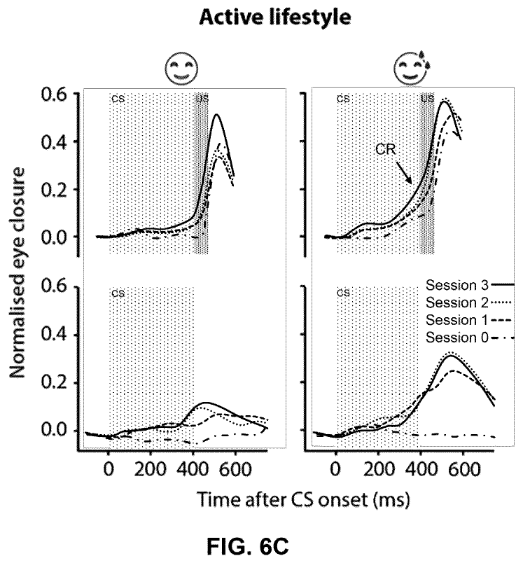

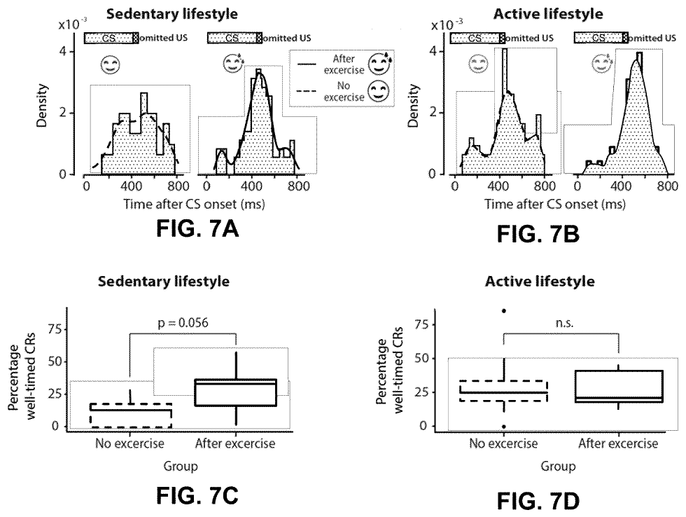

Next it was determined whether aerobic exercise had an effect on the latency to CR peak. For this, CS-only trials were analysed. For all groups, the CR peak times are roughly distributed around the expected onset of the US (see FIGS. 7A and 7B). The latencies to CR peaks did not differ significantly between no-exercise and post-exercise groups for both sedentary (F_{1,16}=0.001, p=0.97) and active (F_{1,16}=1.46, p=0.25, see Table 2) individuals.

The mean percentage of well-timed CRs was also determined for each group (see Table 2, FIGS. 7C and 7D). While the percentage of well-timed CRs was quite low for the sedentary, no-exercise group (see FIG. 7C, mean=11.27%+12.13), the effect of exercise on the percentage of well-timed CRs was neither significant for the sedentary (F_{1,16}=4.25, p=0.056) nor the active group (F_{1,16}=0.19, p=0.67).

### Unconditioned Responses

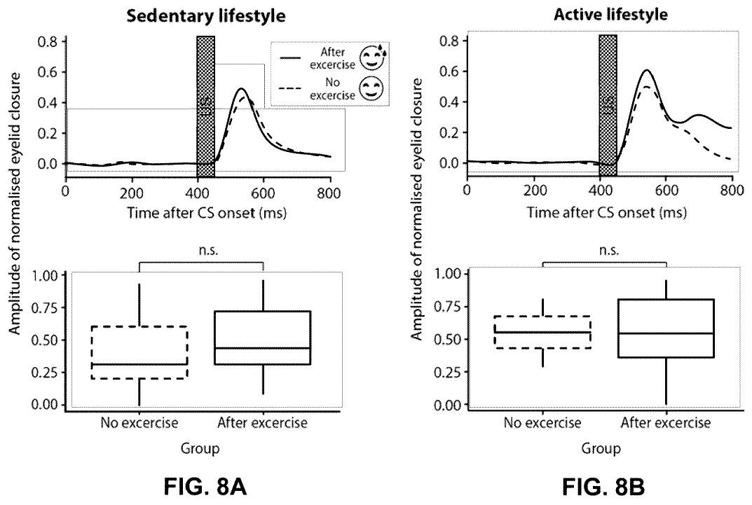

To determine if the effect of aerobic exercise was specific to CRs or more generalised, unconditioned response amplitude was compared between groups. The pre-block and block 1 trials of session 1 were used to determine unconditioned response amplitude, as these data were obtained before onset of the CRs that could in principle influence the amplitude of the unconditioned response. In both the sedentary (F_{1,14}=0.15, p=0.71) and active groups (F_{1,13}=0.11, p=0.75), there was no significant difference in unconditioned response amplitude between the groups that completed the session with or without exercise (see FIGS. 8A and 8B, Table 2).

Aerobic exercise enhanced CR acquisition in a smartphone-mediated eyeblink conditioning paradigm. This effect of exercise was, however, only seen in individuals with an active lifestyle. This finding parallels previous work, where acute exercise enhanced recognition memory in individuals with a prior four week exercise training program, but not in individuals without such a program. Exercise had no major effect on the unconditioned response amplitude or the timing of CR peaks.

Both the active post-exercise and no-exercise groups showed significant CRs in session 1 compared to baseline. Exercise may have a priming effect; reducing the number of practice sessions needed for implicit learning. This enhancing effect of exercise was specific to CRs as the amplitude of the unconditioned responses did not differ between groups. It is proposed that locomotor activity acts directly within the cerebellar cortex to modulate eyeblink conditioning. Locomotor activity signalling via cerebellar mossy fibres (MF) may converge with the CS MF signalling hereby facilitating learning. While exercise may have acted directly within the cerebellar cortex to enhance learning, it is unclear why such an effect would differ for active and sedentary individuals.

The finding that acute exercise facilitates eyeblink conditioning in active but not sedentary individuals may point towards a mechanistic role of neuropeptidergic transmitters and/or neurotrophins. Indeed, both human and animal studies on neuropeptidergic transmitters and neurotrophins show differential effects of acute exercise in active compared to sedentary subjects. Likewise, the dopaminergic, adrenergic and norepinephrinergic pathways, which are all catecholaminergic systems that prominently co-release neuropeptides, are upregulated in humans and animals following exercise. While the proposed role of these neurotransmitters in exercise-induced cognitive benefits are frequently studied, their potential influence on associative procedural learning has received less attention. Despite this, there is evidence for a role of neurotransmitters in cerebellar learning. In rabbits, pharmacological monoamine depletion resulted in a dose-dependent reduction in CRs in an eyeblink conditioning task. Additionally, in rats, cerebellar norepinephrine was shown to be involved in the acquisition of CRs. These findings may extend to humans, where increased levels of norepinephrine following exercise have been associated with improved motor skill acquisition compared to resting controls and where chronic training increased the plasma catecholamine response, compared to no training, after a cycling task.

Similarly, the neurotrophin BDNF may facilitate exercise-induced brain plasticity and memory formation. Notably, BDNF mutant mice show impaired eyeblink conditioning and the impact of tDCS on eyeblink conditioning in humans can depend on BDNF mutations. Moreover, evidence suggests active and sedentary individuals differ in their BDNF response to exercise. A meta-analysis of the effects of exercise on BDNF in humans reported an enhanced BNDF response to acute exercise in active compared to sedentary individuals. Thus it is possible that in this study the acute exercise in the sedentary group was not sufficient to induce the BDNF levels needed to enhance eyeblink conditioning. Together, these findings provide tentative molecular clues as to why in this study aerobic exercise enhanced learning in active but not sedentary individuals.

Unlike the acquisition of CRs, the timing of these responses did not significantly differ across groups. Interestingly, while not significantly different, the percentage of well-timed CRs was slightly higher in the sedentary post-exercise compared to the sedentary no-exercise group.

As disclosed in this example, acute aerobic exercise enhanced the acquisition of learning in an eyeblink conditioning paradigm. Moreover, we found that the effect of exercise differed for active and sedentary individuals. These results confirm in humans what has been shown in animals regarding the facilitatory effects of exercise on eyeblink conditioning. By focusing on a well-characterised learning paradigm, this study contributes to a more objective understanding of how exercise influences the brain.

Thus, for example, by determining a user experienced a statistically significant improvement in CR acquisition over a baseline rate of CR acquisition, the system could determine whether a user has engaged in an effective exercise regime.

As noted previously, the disclosed technique allows the application to correlate the determined values relating to eye movement and eye blinks to various metrics of interest, which could be related to, e.g., emotional engagement with a given video, effectiveness of physical activity, etc. In some embodiments, the method may include calculating a cognitive load based on the eye movement and eye blink values, where the cognitive load can be the metric of interest or one or more proxies for such metric.

In another example, the effect of physical activity can be seen with these systems. As will be understood, non-limiting examples of physical activities that can be considered by the system include exercise, meditation, breathing exercises, sleep, etc. Individuals who are engaged with a cardio or fitness program have a distinct phenotype that can be detected with the disclosed techniques. For example, engaged individuals may be more responsive and have less “noisy” results after a fitness program than non-engaged individuals. Examples of the effect of a physical activity on various metrics can be seen in FIG. 6A-6C.

In some embodiments, the user may be provided a user interface indicating results of the testing. For example, for self-improvement purposes, or for determining a state of alertness when interacting with certain content.

In some embodiments, a user may receive results in a user interface, e.g., on a watch, phone, etc., indicating their level of engagement in a particular fitness activity.

Various modifications may be made to the systems, methods, apparatus, mechanisms, techniques and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined of the claims.

What is claimed is:

1. A method for measuring emotional engagement while watching video or picture content, comprising:
displaying, on a display, a first video or picture content;
while an individual is watching the first video or picture content on the display:
using a camera to capture second video including one or more eyes of the individual; and
while capturing the second video, exposing the individual to one or more visual and/or auditory stimuli;
determining values representing eye closure based on the second video; and
calculating a metric based on the values.

2. The method of claim 1, wherein the method is performed on a single local device.

3. The method of claim 2, wherein the single local device is a desktop computer, laptop computer, mobile phone, or tablet.

4. The method of claim 1, wherein determining values and calculating the metric are performed by one or more remote processing units.

5. The method of claim 1, wherein the display and camera are operably coupled to a headset, the headset being operably coupled to one or more processing units performing the method.

6. The method of claim 5, wherein the headset is a virtual reality (VR) headset.

7. The method of claim 5, wherein the headset is an augmented reality (AR) or mixed reality (MR) headset.

8. The method of claim 1, wherein calculating the metric is performed by a trained machine learning algorithm that has been trained using categorized videos and/or picture content.

9. The method of claim 1, wherein the metric is based at least partially on detected alpha startle responses.

10. The method of claim 1, further comprising receiving first information from one or more remote processing units, the first information including the first video or picture content.

11. The method of claim 10, wherein the first information further includes the one or more visual and/or auditory stimuli, values representative of the one or more visual and/or auditory stimuli, or both.

12. The method of claim 1, further comprising sending second information to one or more remote processing units, the second information including the second video.

13. The method of claim 1, further comprising displaying the metric to the individual.

14. The method of claim 1, wherein the metric is emotional engagement, and the metric is determined based on a startle response.

15. The method of claim 1, wherein the metric is an impact of physical activity, and the metric is determined based on eyeblink conditioning.

16. A system for measuring emotional engagement while watching video or picture content, comprising:
a display;
a camera;
a speaker;
a memory;
one or more processing units operably coupled to the display, camera, speaker, and memory; and
a non-transitory computer-readable storage medium comprising instructions that, when executed by the one or more processing units, cause the one or more processing units to, collectively:
display, on the display, first video or picture content;
while an individual is watching the first video or picture content on the display:
cause the camera to capture second video including one or more eyes of the individual; and
while capturing the second video, expose the individual to one or more visual and/or auditory stimuli;
determine values representing eye closure based on the second video including one or more eyes of the individual; and
calculate a metric based on the values.

17. The system of claim 16, wherein the system is configured as a desktop computer, laptop computer, mobile phone, or tablet.

18. The system of claim 16, wherein the one or more processing units includes one or more local processing units and one or more remote processing units, where the one or more remote processing units are configured to, collectively, determine values and calculate the metric.

19. The system of claim 16, wherein the display and camera are operably coupled to a headset, the headset being operably coupled to the one or more processing units.

20. The system of claim 19, wherein the headset is a virtual reality (VR) headset.

21. The system of claim 19, wherein the headset is an augmented reality (AR) or mixed reality (MR) headset.
